(19) United States
(12) Patent Application Publication
(10) Pub. No.: US 2010/0184045 A1
(43) Pub. Date: Jul. 22, 2010

(54) Methods for sequencing degraded or modified nucleic acids

(75) Inventor: John F. Thompson, Warwick, RI (US)

Correspondence Address: BROWN RUDNICK LLP, ONE FINANCIAL CENTER, BOSTON, MA, 02111, US

(73) Assignee: Helicos Biosciences Corporation, Cambridge, MA (US)

(21) Appl. No.: 12/586,539

(22) Filed: Sep. 23, 2009

Related U.S. Application Data
(60) Provisional application No. 61/099,389, filed on Sep. 23, 2008.

(51) Int. Cl. C12Q 1/68

(52) U.S. Cl. 435/6

(57) ABSTRACT

The invention provides methods and compositions for sequencing DNA or RNA samples that would be impossible to do via standard means. Samples that are part of mixtures or are degraded or modified may be sequenced so that the individual from whom the sample originated can be determined or useful biological information can be associated with the sample. Methods are described that allow high efficiency sequencing of degraded nucleic acid samples such as are typically found with FFPE. Samples from severely degraded sources or that have been treated with preservatives such as formalin may be sequenced. In addition to permitting identification of samples, information about disease or treatment status may also be determined.

### RELATED APPLICATION

The present invention is related to and claims the benefit of U.S. provisional patent application Ser. No. 61/099,389, filed Sep. 23, 2008, the contents of which are incorporated by reference herein in their entirety.

### TECHNICAL FIELD

The invention is in the field of molecular biology and relates to methods for nucleic acid analysis. In particular, the invention relates to methods of nucleic acid sequencing that are useful for samples that are degraded or otherwise impure.

### BACKGROUND

The value of high throughput, single molecule sequencing for deriving important information regarding many aspects of biology, disease, and identification of individuals has been shown repeatedly. With many technologies, sample preparation prior to sequencing is a significant issue, requiring extensive expertise and skills and still failing to yield DNA or RNA of sufficient quality a large fraction of the time. Methods that simplify sample preparation prior to sequencing would be valuable in saving time and resources and also generate a higher yield of successfully sequenceable samples. Furthermore, some samples such as from Formalin Fixed Paraffin Embedded (FFPE) blocks, from which sequence information is desired, are of poor quality. Any copying or amplification of such samples runs the risk of failing due to modifications in the nucleic acid at intervals that prevent a polymerase or transcriptase from reading through the modification. Techniques that require minimal or no copying of the DNA or RNA prior to sequencing should yield a higher success rate and less chance of biasing the sequencing results. Such low quality samples include but are not limited to DNA/RNA stored as FFPE samples, forensic samples present in decomposing remains, and archeological samples that have been stored for many years. In addition to human samples, the nucleic acid from many other species is also valuable to sequence and may encounter similar issues.

### SUMMARY OF THE INVENTION

The invention provides methods and compositions for high throughput, single molecule sequencing-by-synthesis of nucleic acid in a manner that requires minimal sample preparation, and allows use of degraded or modified DNA or RNA. These methods may be used with samples that are too degraded to be sequenced using standard techniques. For example, FFPE samples or samples from bodies or tissues that have undergone extensive decomposition can be potentially identified using this sequencing approach. Human nucleic acid from such samples could be sequenced either for identification or to better characterize the disease/treatment status of the individual from whom the sample originated.

In preferred embodiments, the methods of the invention involve the addition of a capture region that is added to the 3′ end of the nucleic acid to be sequenced. The addition may be made by ligation or polymerization but is done without the need for amplification of the nucleic acid to be sequenced.

The nucleic acids to be sequenced may be DNA or RNA. If RNA, it may be copied with a reverse transcriptase first, or, in some embodiments, sequenced directly with reverse transcriptase. If copied first to generate cDNA, such copying may be primed by short primers including but not limited to random oligomers, oligomers selected to avoid priming ribosomal RNA or other frequent RNAs of low interest, or oligomers selected to specifically prime genes or gene families of interest. RNA may be treated before or after copying to selectively remove ribosomal RNA or other RNAs of low interest as by hybridization to antisense ribosomal RNA.

The DNA, RNA or cDNA to be sequenced from FFPE or other low quality source will be much shorter than standard DNA samples and may contain base modifications, breaks in the phosphodiester backbone, missing or severely altered bases, or crosslinks that impede amplification and other typical means of working with the nucleic acid. Some or all of these modifications would negatively affect the ability to sequence DNA using other methodologies. To avoid such problems, sample manipulation must be kept to a minimum. To this end, the nucleic acid to be sequenced will be modified at only the 3′ end in such a manner as to limit the impact of any upstream modifications on the 3′ manipulation. The 3′ manipulation is then used as a capture sequence and primer for single molecule sequencing which can be carried out up to the first point within the nucleic acid that bears a fatal modification. In one preferred embodiment, the nucleic acid is labeled at the 3′ using terminal transferase and a single dNTP so that a homopolymer is generated of sufficient length that specific hybridization and priming are possible. In another preferred embodiment, an oligonucleotide is ligated to the end of the nucleic acid. The target nucleic acid is hybridized using conditions that allow some level of specificity for the sequence present in the target. After hybridization, each capture oligonucleotide is extended in a template-dependent manner with a polymerase or transcriptase in the presence of detectably labeled nucleotides, or analogs, and the incorporated nucleotide(s) are detected so that the sequence of the original targeted nucleic acid is determined.

Accordingly, the methods of invention include:
a) attaching a specific sequence to the 3′ end of the target nucleic acid,
b) anchoring to a substrate one or more capture oligonucleotides, complementary to the sequence of interest that is placed at the 3′ end of the FFPE or degraded DNA sample,
c) hybridizing the target nucleic acid to the capture oligonucleotide(s) under annealing conditions,
d) sequencing the anchored nucleic acid strands, and
e) using that information for identification, determining the abundance, or variations of the sequence.

In some embodiments, the target nucleic acid may be analyzed without being copied. In some embodiments the sequencing is performed using sequencing-by-synthesis. In preferred embodiments, the sequencing is performed at single molecule resolution. Several sequencing by synthesis methods are known in the art.

An example of asynchronous single molecule sequencing-by-synthesis is illustrated in FIG. 1. As shown, oligonucleotides 30-50 bases in length are covalently anchored at the 5′ end to glass cover slips. These anchored strands perform two functions. First, they act as capture sites for the target template strands, if the templates are configured with capture tails complementary to the surface bound oligonucleotides. They also act as primers for the template-directed primer extension that forms the basis of the sequence reading. The capture primers are a fixed position site for sequence determination. Each cycle consists of adding the polymerase-labeled nucleotide analog mixture, rinsing, optically imaging the field containing millions of active primer template duplexes, and chemically cleaving the dye-linker to remove the dye. The labeled nucleotides are added either individually in a cycle or if the detectable moiety is spectrally resolvable more than one nucleotide can be added per cycle. The nucleotide analogs are such that they add only once per strand/cycle, e.g., a reversible terminator. The cycle (synthesis, detection, and dye removal) is repeated up to 25, 50, 100 times and, possibly, more.

The real-time single molecule sequencing-by-synthesis technologies rely on the detection of fluorescent nucleotides as they are incorporated into a nascent strand of DNA that is complementary to the template being sequenced. This type of detection depends upon the ability of the imaging system to differentiate which of the four spectrally resolvable fluorescent nucleotides in the polymerase-labeled nucleotide mixture incorporates as the polymerase copies the template in near real-time.

Four major high-throughput sequencing platforms are currently available: the Genome Sequencers from Roche/454 Life Sciences (Margulies et al. (2005) Nature, 437:376-380; U.S. Pat. Nos. 6,274,320; 6,258,568; 6,210,891), the 1G Analyzer from Illumina/Solexa (Bennett et al. (2005) Pharmacogenomics, 6:373-382), the SOLiD system from Applied Biosystems (solid.appliedbiosystems.com), and the Heliscope™ system from Helicos Biosciences (see, e.g., U.S. Patent App. Pub. No. 2007/0070349 and the illustration in FIG. 1). Although these new technologies are significantly cheaper compared to the traditional methods, such as gel/capillary Gilbert-Sanger sequencing, the sequence reads produced by the new technologies are generally much shorter (˜25-40 vs. ˜500-700 bases). For example, the average read lengths on the four major platforms are currently as follows: Roche/454, 250 bases (depending on the organism); Illumina/Solexa, 25 bases; SoliD, 35 bases; Heliscope, 25 bases.

### BRIEF DESCRIPTION OF THE DRAWINGS

FIG. 1 is a graph show an RNA analysis comparison between fresh frozen samples and FFPE samples. The total count for fresh frozen was 0.5M and the total count for FFPE was 0.2M. The highest expressers were: rRNA at 54% to 59%; and mitochondrial genes at 5% to 7%. The correlation was 0.984 (driven by high express), and the dynamic range was 4 to 5 orders of magnitude.

FIG. 2 is a graph showing classification of select genes as low, medium, and high expressors. The graph shows comparison by three categories. There was agreement on 23,889 (83%) of 28,829 transcripts, and no radical disagreements.

FIG. 3 is a graph showing a comparison of fresh and FFPE sample variation. About 85% of transcripts had an abundance≧10 tpm, which demonstrates less than a 4-fold difference between the two samples. About 60% of transcripts had an abundance of 50 tpm, which demonstrates less than a 2-fold difference between the two samples.

### DETAILED DESCRIPTION OF THE INVENTION

The invention provides methods and compositions for analyzing the sequence and/or modification pattern of a target nucleic acid. In general, the methods of the invention include:
a) modifying the 3′ end of the nucleic acid of interest via polymerization or ligation of a specific sequence such that it is complementary to the capture oligonucleotide,
b) anchoring the capture oligonucleotides to a substrate,
c) hybridizing the target nucleic acid to the substrate,
d) sequencing at least a portion of the target nucleic acid, and
e) identifying an individual or a pattern of sequences useful for understanding biological processes.

In more specific embodiments, methods of the invention include:
a) using nucleic acid from a FFPE sample,
b) tailing the 3′ end of the nucleic acid with terminal transferase and dATP,
c) hybridizing to a capture sequence dT50 on a surface, and
d) sequencing by single molecule sequence-by-synthesis so as to sequence at least a portion of the target nucleic acid.

Some embodiments further include determining the efficiency of the detection for the detected target nucleic acid, e.g., by spiking the starting sample with a known amount of detectable target nucleic acid.

Additional methods and compositions of the invention are described in detail below.

### Capture Oligonucleotides

In one embodiment, the capture oligonucleotide comprise a homopolymer complementary to a homopolymer at or near the end of a target sequence and oriented in a manner that template-dependent extension at the 3′ end would extend into the target sequence. The length of the capture oligonucleotide may vary. In general, it should be of sufficient length to provide a relatively stable structure. For example, the double-stranded region, when hybridized to nucleic acid, may be 10-100, 20-75, 40-75, or about 50 nucleotides long when composed of oligo dT. These capture oligonucleotides would preferably be modified at the 5′ end to enable anchoring on the substrate. The anchoring may be either covalent or non-covalent. Examples of non-covalent are via a binding pair, e.g., biotin:streptavidin or through the polymerase. Any component, e.g., oligonucleotide, nucleic acid or the polymerase may be the moiety which anchors the complex to the substrate. In a preferred embodiment, anchoring is accomplished by the covalent attachment of a 5′-amine modified oligonucleotide to an epoxide coated planar substrate, e.g., glass or silicon.

### Target Nucleic Acid Samples

Target nucleic acid samples can come from a variety of sources and consist of DNA/RNA of a quality and purity typically used for sequencing, consist of DNA/RNA that is part of a mixture, or can consist of DNA/RNA that is much more heavily modified or degraded than is typically possible to use for sequencing. If the nucleic acid is longer than 300 nucleotides, it may be desirable to further fragment the nucleic acid to a size of 25-300 nucleotides by a nuclease digestion or shearing technique but this is not generally necessary with degraded or FFPE samples. If the nucleic acid is already less than 300 nucleotides due to degradation or sample handling, it can be used directly. Some samples, due to age or storage conditions, are very short or contain modified or deleted bases. Normally, these cannot be used for sequencing by other methods due to the extensive amplification required. By directly capturing the nucleic acid for single molecule sequencing-by-synthesis, those heavily modified strands can be sequenced up to the point of a defect (from the perspective of the polymerase/transcriptase). In addition to the sequence information obtained, such points of termination may also yield important information if those modifications are specific.

In some embodiments, the methods of the invention include obtaining nucleic acids from whole organisms, organs, tissues, cells, or biological fluids/waste products (urine, blood, stool, etc.) and these samples may be from different stages of development, differentiation, or disease state, and from different species (e.g., human and non-human animals, primates, rodents, plants). Various methods for extraction of nucleic acids from biological samples are known (see, e.g., Nucleic Acids Isolation Methods, Bowein (ed.), American Scientific Publishers, 2002). The sample may be pre-purified prior to the addition to the capture oligonucleotides, for example, by ethanol precipitation of nucleic acid or other suitable methods.

The invention further provides compositions for use in the methods of the invention, including individual capture oligonucleotides systems, and kits.

### Sequencing Platforms

A number of initiatives are currently underway to obtain sequence information directly from millions of individual molecules of DNA or RNA in parallel. Real-time single molecule sequencing-by-synthesis technologies rely on the detection of fluorescent nucleotides as they are incorporated into a nascent strand of DNA that is complementary to the template being sequenced. In one method, oligonucleotides 25-50 bases in length are covalently anchored at the 5′ end to glass cover slips. These anchored strands perform two functions. First, they act as capture sites for the target template strands if the templates are configured with capture tails complementary to the surface-bound oligonucleotides. They also act as primers for the template directed primer extension that forms the basis of the sequence reading. The capture primers function as a fixed position site for sequence determination using multiple cycles of synthesis, detection, and chemical cleavage of the dye-linker to remove the dye. Each cycle consists of adding the polymerase/labeled nucleotide mixture, rinsing, imaging and cleavage of dye. In an alternative method, polymerase is modified with a fluorescent donor molecule and immobilized on a glass slide, while each nucleotide is color-coded with an acceptor fluorescent moiety attached to a gamma-phosphate. The system detects the interaction between a fluorescently-tagged polymerase and a fluorescently modified nucleotide as the nucleotide becomes incorporated into the de novo chain. Other sequencing-by-synthesis technologies also exist.

The invention can be used on any suitable single molecule sequencing-by-synthesis platform. As described above, one sequencing-by-synthesis platforms that is currently available is the Heliscope system from Helicos Biosciences. Single molecule sequencing-by-synthesis platforms have also been described by Pacific BioSciences, and VisiGen Biotechnologies. These and other single molecule systems could potentially be used in the methods of the invention.

The substrate may be, for example, a glass surface such as described in, e.g., US Patent App. Pub. No. 2007/0070349. The surface may be coated with an epoxide, polyelectrolyte multilayer, or other coating suitable to bind nucleic acids. In preferred embodiments, the surface is coated with epoxide and a complement of the capture sequence is attached via an amine linkage. The surface may be derivatized with avidin or streptavidin, which can be used to attach to a biotin-bearing target nucleic acid. Alternatively, other coupling pairs, such as antigen/antibody or receptor/ligand pairs, may be used. The surface may be passivated in order to reduce background. Passivation of the epoxide surface can be accomplished by exposing the surface to a molecule that attaches to the open epoxide ring, e.g., amines, phosphates, and detergents.

Subsequent to the capture, the sequence may be analyzed, for example, by single molecule detection sequence-by-synthesis, e.g., as described in the Example and in U.S. Pat. No. 7,283,337. In sequencing-by-synthesis, the surface-anchored complex is exposed to a plurality of detectable labeled nucleotide triphosphates in the presence of polymerase. The sequence of the template is determined by recording the order of labeled nucleotides incorporated into the 3′ end of the growing chain. This can be done in real time or can be done in a step-and-repeat mode. For real-time analysis, different spectrally resolvable, optical labels to each nucleotide may be incorporated and multiple lasers may be utilized for stimulation of incorporated nucleotides.

### Examples

The following Examples provide illustrative embodiments of the invention and do not limit the invention in any way.

### Example 1

### FFPE Samples

Nucleic acid can be purified from FFPE blocks using any standard technique. The nucleic acid can be used directly or, in the case of RNA, may be reverse transcribed to cDNA prior to further manipulation. Reverse transcription can be primed with a variety of different oligonucleotides or self-priming polymerases. Short oligonucleotides consisting of mixed hexamers are frequently used and may be a completely random assortment or may be selected to include only a specific subset of oligonucleotides so as to preferentially prime RNA species of higher interest. The RNA may be treated before or after cDNA synthesis so as to remove less interesting RNA molecules such as ribosomal or mitochondrial RNA. The DNA, RNA, or cDNA samples may be reacted with terminal transferase and dATP or other suitable nucleotide triphosphate to add a tail at the 3′ end sufficiently long to allow specific hybridization to a complementary homopolymer capture sequence. This reaction may be controlled by addition of a carrier nucleic acid that will have properties that allow its selective removal prior to sequencing. In this fashion, the target nucleic acid concentration may have a wide range of concentrations and still be effectively manipulated. After addition of the homopolymer at the 3′ end of the target nucleic acid, it can then be hybridized to complementary capture oligonucleotides and sequenced without need for further copying or amplification.

### Example 2

### Single Molecule Sequencing

Epoxide-coated glass slides are prepared for oligo attachment. Epoxide functionalized 40 mm diameter #1.5 glass cover slips (slides) are obtained from Erie Scientific (Salem, N.H.). The slides are preconditioned by soaking in 3×SSC for 15 minutes at 37° C. Next, a 500-pM aliquot of 5′ aminated capture oligonucleotide (oligo dT(50)) is incubated with each slide for 30 minutes at room temperature in a volume of 80 ml. The slides are then treated with phosphate (1 M) for 4 hours at room temperature in order to passivate the surface. Slides are then stored in 20 mM Tris, 100 mM NaCl, 0.001% Triton® X-100, pH 8.0 at 4° C. until they are used for sequencing.

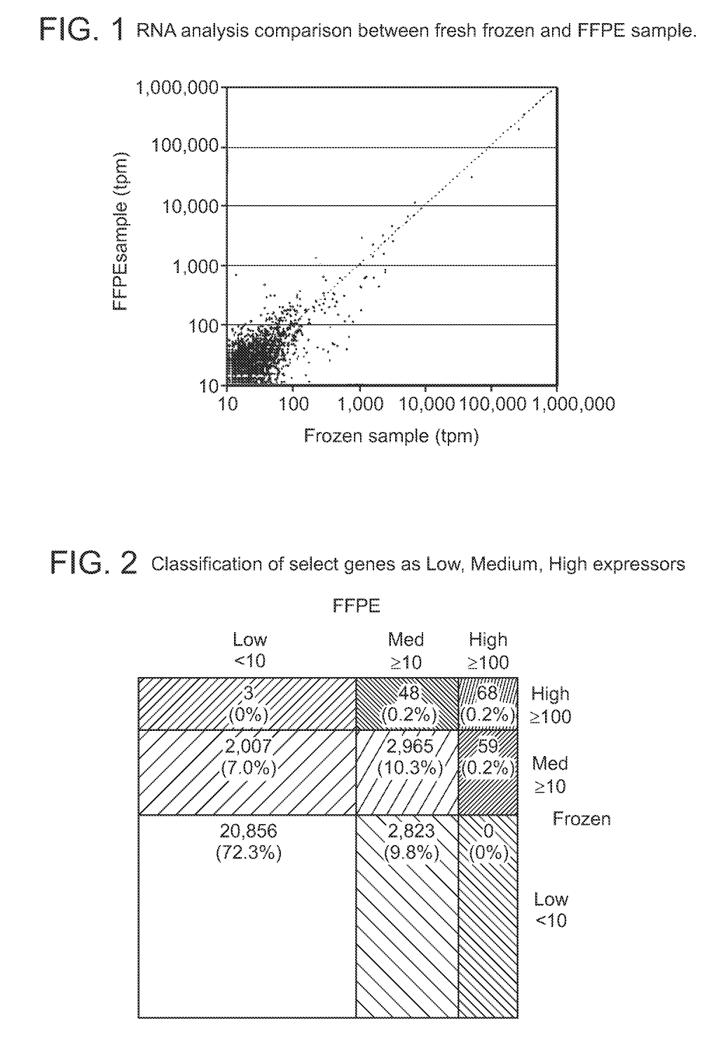

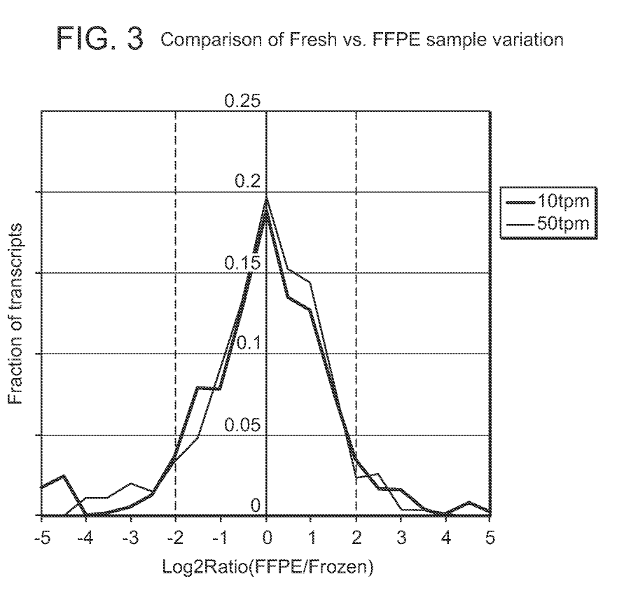

For the illustration of the sequencing process, see, e.g., U.S. patent application Ser. Nos. 12/043,033 (Xie et al. filed Mar. 5, 2008) and U.S. Ser. No. 12/113,501 (Xie et al. filed May 1, 2008) (e.g., FIGS. 1A and 1B). For sequencing, the slide is assembled into a 25 channel flow cell using a 50-μm thick gasket. The flow cell is placed into a Heliscope™ Sample Loader (Helicos BioSciences Corporation). A passive vacuum is built into the apparatus and is used to pull fluid across the flow cell. The flow cell is then rinsed with 150 mM HEPES/150 mM NaCl, pH 7.0 (“HEPES/NaCl”) and equilibrated to a temperature of 50° C. Separately, the nucleic acid to be sequenced is sheared to approximately 200-500 bases (Covaris), polyA tailed (50-70 ave. number dA's) using dATP and terminal transferase (NEB), 3′end labeled with Cy5-ddUTP (PerkinElmer), and then diluted in 3×SSC to a final concentration of approximately 200 pM. A 100-μL aliquot is placed in one or more channels of the flow cell and incubated on the slide for 15 minutes. After incubation, the temperature of the flow cell is then reduced to 37° C. and the flow cell is rinsed with 1×SSC/150 mM HEPES/0.1% SDS, pH 7.0 (“SSC/HEPES/SDS”) followed by HEPES/NaCl. The resulting slide contains the primer template duplex randomly bound to the glass surface. Since the polyA/oligoT sequences are able to slide, the primer templates are filled and locked by firstly incubating the surface with Klenow exo+, TTP, in reaction buffer (NEB), washing thoroughly with HEPES/NaCl, and then incubating with Klenow exo+, dATP/dCTP/dGTP, in reaction buffer (NEB). The slide is washed thoroughly again using the HEPES/NaCl to remove all traces of the dNTPs before initiating the actual sequencing by synthesis process. The temperature of the flow cell is maintained at 37° C. for sequencing and the objective is brought into contact with the flow cell.

Further, Virtual Terminator™ nucleotide analogs of 2′-deoxycytosine triphosphate, 2′-deoxyguanidine triphosphate, 2′-deoxyadenine triphosphate, and 2′-deoxyuracil triphosphate, each having a cleavable cyanine-5 label (at the 7-deaza position for ATP and GTP and at the C5 position for CTP and UTP, see, e.g., U.S. patent application Ser. Nos. 111803,339 (Siddiqi et at. filed May 14, 2007) and U.S. Ser. No. 11/603,945 (Siddiqi et at. filed Nov. 22,2006), are stored separately in the buffer containing 20 mM Tris-HCl, pH 8.8, 50 μM MnSO₄, 10 mM (NH)₂SO₄, 10 mM KCl, 10 mM NaCl and 0.1% Triton X-100, and 50 U/mL Klenow exo-polymerase (NEB).

Sequencing proceeds as follows. The flow cell is placed on a movable stage that is part of a high-efficiency fluorescence imaging system Heliscope™ Single Molecule Sequencer (Helicos BioSciences Corporation). First, initial imaging is used to determine the positions of duplex on the epoxide surface. The Cy5 label attached to the nucleic acid template fragments is imaged by excitation using a laser tuned to 635 nm radiation in order to establish duplex position. For each slide only single fluorescent molecules that are imaged in this step are counted. Next, the cyanine-5 label is cleaved off incorporated template by introduction into the flow cell of 50 mM TCEP/250 mM Tris, pH 7.6/100 mM NaCl (“TCEP solution”) for 5 minutes, after which the flow cell is rinsed with SSC/HEPES/SDS and HEPES/NaCl. The template is capped with 50 mM iodoacetamide/100 mM Tris, pH 9.0/100 mM NaCl (“Iodoacetamide solution”) for 5 minutes followed by rinse with SSC/HEPES/SDS and HEPES/NaCl. Imaging of incorporated nucleotides as described below is accomplished by excitation of a cyanine-5 dye using a 635-nm radiation laser. 100 nM Cy5-dCTP is placed into the flow cell and exposed to the slide for 2 minutes. After incubation, the slide is rinsed in SSC/HEPES/SDS, followed by HEPES/NaCl. An oxygen scavenger containing 30% acetonitrile and scavenger buffer (100 mM HEPES, 67 mM NaCl, 25 mM MES, 12 mM Trolox, 5 mM DABCO, 80 mM glucose, 5 mM NaI, and 0.1 U/μL glucose oxidase (USB), pH 7.0) is next added. The slide is then imaged (100-1000 frames) for 50-100 milliseconds at 635 nm. The positions having detectable fluorescence are recorded. After imaging, the flow cell is rinsed with SSC/HEPES/SDS and HEPES/NaCl. Next, the cyanine-5 label is cleaved off incorporated dCTP by introduction into the flow cell of TCEP solution for 5 minutes, after which the flow cell is rinsed with SSC/HEPES/SDS and HEPES/NaCl. The remaining nucleotide is capped with Iodoacetamide solution for 5 minutes followed by rinse with SSC/HEPES/SDS and HEPES/NaCl. Optionally, the scavenger is applied again in the manner described above, and the slide is again imaged to determine the effectiveness of the cleave/cap steps and to identify non-incorporated fluorescent objects.

The procedure described above is then conducted 100 nM Cy5-dATP, followed by 100 nM Cy5-dGTP, and finally 100 nM Cy5-dUTP. Uridine may be used instead of Thymidine due to the fact that the Cy5 label is incorporated at the position normally occupied by the methyl group in Thymidine triphosphate, thus turning the dTTP into dUTP. The procedure (expose to nucleotide, polymerase, rinse, scavenger, image, rinse, cleave, rinse, cap, rinse, scavenger, final image) is repeated for a total of 80-120 cycles.

Once the desired number of cycles is completed, the image stack data (i.e., the single-molecule sequences obtained from the various surface-bound duplexes) are aligned to produce the individual sequence reads. The individual single molecule sequence read lengths obtained range from 2 to 50+ consecutive nucleotides. Only the individual single molecule sequence read lengths above some predetermined cut-off depending upon the nature of the sample, e.g. greater than 20 and above, are analyzed using the method of the invention.

All publications, patents, patent applications, and biological sequences cited in this disclosure are incorporated by reference in their entirety.

1. A method for determining the sequence of a target nucleic acid, wherein the target nucleic acid is modified or degraded, the method comprising:
a) isolating the target nucleic acid,
b) modifying the target nucleic acid on the 3′ end, 5′ end, or both,
c) anchoring the target nucleic acid to a substrate, and
d) sequencing at least a portion of the target nucleic acid.

2. The method of claim 1, wherein the modification or degradation is due to treatment with a preservative(s).

3. The method of claim 2, wherein the preservative is formalin.

4. The method of claim 3, wherein the formalin preservative results from formalin fixed paraffin embedding of tissue samples.

5. The method of claim 1, wherein the nucleic acid is DNA or RNA.

6. The method of claim 5, wherein the DNA is cDNA.

7. The method of claim 1, wherein the modified target nucleic acid is anchored directly or indirectly to the substrate.

8. The method of claim 7, wherein the anchoring is by hybridization.

9. The method of claim 7, wherein the anchoring is via a complex with a polymerase.

10. The method of claim 1, wherein the sequencing in step d) is sequencing by synthesis.

11. The method of claim 10, wherein the sequencing by synthesis method detects single molecules which are individually optically resolvable.

12. The method of claim 1, wherein the sequencing in step d) is sequencing by ligation.

13. The method of claim 12, wherein the sequencing by ligation comprises detecting single molecules which are individually optically resolvable.

14. A method for determining the sequence of a target nucleic acid, wherein the target nucleic acid is modified or degraded, the method comprising:
a) isolating the target nucleic acid,
b) modifying the target nucleic acid by adding a specific sequence to the 3′-end,
c) anchoring the target nucleic acid to a substrate coated with an oligonucleotide complementary, at least in part, to the sequence added in b), and
d) sequencing at least a portion of the target nucleic acid.

15. The method of claim 14, wherein the 3′-specific sequence is>20 bases in length or longer.

16. The method of claim 15, wherein a 3′-specific sequence is added via ligation.

17. The method of claim 15, wherein a 3′-specific sequence is added using a transferase and a single dNTP.

18. The method of claim 14, wherein the target nucleic acid is anchored directly or indirectly to the substrate.

19. The method of claim 18, wherein the anchoring is by hybridization.

20. The method of claim 18, wherein the anchoring is via a complex with a polymerase.

21. The method of claim 14, wherein the sequencing in step d) is sequencing by synthesis.

22. The method of claim 21, wherein the sequencing by synthesis comprises detecting single molecules which are individually optically resolvable.

23. The method of claim 14, wherein the sequencing in step d) is sequencing by ligation.

24. The method of claim 23, wherein the sequencing by ligation comprises detecting single molecules which are individually optically resolvable.

25. A method for determining the expression level of genes within a sample wherein the target nucleic acid is modified or degraded RNA, the method comprising:
a. isolating the RNA,
b. making cDNA,
c. modifying the cDNA by adding a sequence specific to the 3′-end,
d. anchoring the modified cDNA to a substrate coated with an oligonucleotide complementary, at least in part, to the sequence added in b),
e. sequence at least a portion of the cDNA,
f. assigning sequences to specific genes, and
g. determining the observation frequency of observed genes.

26. The method of claim 25, wherein the nucleic acid is total RNA.

27. The method of claim 25, wherein the nucleic acid is mRNA.

28. The method of claim 25, wherein the 3′-specific sequence is>20 bases in length.

29. The method of claim 25, wherein a 3′-specific sequence is added via ligation.

30. The method of claim 25, wherein a 3′-specific sequence is added using a transferase and a single dNTP.

31. The method of claim 25, wherein the target nucleic acid is anchored directly or indirectly to the substrate.

32. The method of claim 31, wherein the anchoring is by hybridization.

33. The method of claim 31, wherein the anchoring is via a complex with a polymerase.

34. The method of claim 25, wherein the sequencing in step d) is sequencing by synthesis.

35. The method of claim 34, wherein the sequencing by synthesis comprises detecting single molecules which are individually optically resolvable.

36. The method of claim 25, wherein the sequencing in step d) is sequencing by ligation.

37. The method of claim 36, wherein the sequencing by ligation comprises detecting single molecules which are individually optically resolvable.

38. The method of claim 1, wherein the method further comprises determining identity(s) of the individual(s) from whom the target nucleic acid originated.

39. The method of claim 1, wherein the method further comprises assessing or predicting the status of an individual's health, or response to treatment.

40. The method of claim 1, wherein the method further comprises determining polymorphisms in the sequence due to substitutions, insertions, deletions, or variations in copy number.

41. The method of claim 1, wherein the method further comprises using a sample originating from a mixture of sources, such as human and bacteria.

42. The method of claim 1, wherein the target nucleic acid is not amplified prior to sequencing.
